(19) United States
(12) Patent Application Publication
(10) Pub. No.: US 2025/0312769 A1
(43) Pub. Date: Oct. 9, 2025

(54) LONG SHELF-LIFE SORBENT CARTRIDGE

(71) Applicant: AWAK TECHNOLOGIES PTE LTD, Singapore (SG)

(72) Inventors: Suresha BELUR VENKATARAYA, Singapore (SG); Mandar Manohar GORI, Singapore (SG); Sanjay Kumar SINGH, Singapore (SG); Joel Preetham FERNANDES, Singapore (SG); Marcin Bartlomiej PAWLAK, Singapore (SG); Sridhar CHIRUMARRY, Singapore (SG); Vinod Kumar GADI, Singapore (SG); Peter HAYWOOD, Singapore (SG)

(73) Assignee: AWAK TECHNOLOGIES PTE LTD, Singapore (SG)

(21) Appl. No.: 18/865,435

(22) PCT Filed: May 18, 2023
(86) PCT No.: PCT/SG2023/050340

§ 371 Date: Nov. 13, 2024

(30) Foreign Application Priority Data
May 19, 2022 (SG) 10202205247R

(51) Int. Cl. B01J 20/20; A61M 1/16; B01J 20/02; B01J 20/06; B01J 20/28; B01J 39/10; B01J 39/12; B01J 47/04

(52) U.S. Cl. CPC B01J 20/20; A61M 1/1696; B01J 20/0211; B01J 20/0292; B01J 20/06; B01J 20/28052; B01J 39/10; B01J 39/12; B01J 47/04; B01J 2220/42; B01J 2220/62

(57) ABSTRACT

Sorbent cartridges having an improved shelf life comprising multiple layers of sorbent materials wherein moisture levels in the multiple layers of sorbent materials are controlled, and wherein a first layer of the sorbent cartridge comprises activated carbon and a second layer in direct contact with the first layer comprises one or both of hydrous zirconium oxide and zirconium phosphate.

### FIELD OF THE INVENTION

The invention relates to a sorbent cartridge having a long shelf life. The improved shelf life is achieved by providing the sorbent in the form of layers having a minimal difference in moisture content between layers.

### BACKGROUND

The listing or discussion of a prior-published document in this specification should not necessarily be taken as an acknowledgement that the document is part of the state of the art or is common general knowledge.

Sorbents are useful in kidney dialysis, specifically in the removal of toxins present in dialysate. Sorbent cartridges involve a number of components, for example ion exchangers such as zirconium phosphate (ZP) and hydrous zirconium oxide (HZO); activated carbon (AC); and a uremic toxin removing component such as (immobilised) urease. The various components in a sorbent are often provided as parts of distinct layers within a cartridge. A cartridge comprising a sorbent is known as a sorbent cartridge.

Patients in need of dialysis often undergo treatment at home overnight, since this allows them to live a relatively normal life during the day and avoids frequent trips to a dialysis centre. In order to do this, the patient requires a dialysis device and all necessary materials for dialysis, such as dialysis fluid and sorbent cartridges, to be stored at their home. It is convenient for patients to be able to store these components for a long time, since this allows them to receive bulk deliveries less frequently.

A problem associated with layered sorbent cartridges is that the layers may separate or crack, and other imperfections or non-uniformity of the sorbent may develop during storage (e.g. after as little as two weeks). Gaps formed by such layer separation or cracking, as well as any imperfections or non-uniformity, will drastically affect dialysis fluid flow through a sorbent, thereby reducing the effectiveness of the sorbent at removing toxins from dialysate, compromising the effectiveness of treatment.

There is therefore a need for sorbent cartridges that may be stably stored for periods of at least 8 weeks and preferably for up to, or beyond, 12 months.

### SUMMARY OF THE INVENTION

The moisture content of the ion exchanging materials in a sorbent plays an important role in the effectiveness of ion exchange. For example, the inventors have found that the ion exchange efficacy of zirconium phosphate and zirconium oxide may be optimised when their moisture content is around 20-23 wt. % and 35-40 wt. %, respectively. In contrast, the inventors have found that the moisture content of the activated carbon layer, which is another core sorbent component, is usually provided with a much lower moisture content of around 8 wt. %.

The inventors have surprisingly found that when the difference in moisture levels between zirconium phosphate/zirconium oxide/activated carbon layers in a sorbent is minimised, the sorbent is stable for a much longer time. This may be achieved without significantly affecting the ion exchange capacity of the sorbent. Without wishing to be bound by theory, it is believed that the substantial difference in moisture level results in layer separation of sorbents having adjacent zirconium phosphate/zirconium oxide/activated carbon layers during longer term storage and so minimising this difference obviates this issue.

The invention therefore provides the following numbered clauses.

1. A sorbent cartridge comprising:
a first layer comprising activated carbon; and
a second layer in direct contact with the first layer, the second layer comprising
one or both of hydrous zirconium oxide and zirconium phosphate,

wherein:
the first layer has a moisture content of X wt. %;
the second layer has a moisture content of Y wt. %; and
the difference in the moisture content of the first layer and the second layer is less than or equal to 8 wt. %.

2. The sorbent cartridge according to Clause 1, wherein the second layer comprises both hydrous zirconium oxide and zirconium phosphate.

3. The sorbent cartridge according to Clause 2, wherein the second layer comprises a homogenous mixture of hydrous zirconium oxide and zirconium phosphate.

4. The sorbent cartridge according to Clause 1, wherein:
the second layer comprises one of zirconium phosphate and hydrous zirconium oxide;
the sorbent cartridge further comprises a third layer comprising the other of zirconium phosphate and hydrous zirconium that is not present in the second layer;
the third layer has a moisture content of Z wt. %; and
the difference in the moisture content of the second layer and the third layer is less than or equal to 8 wt. %.

5. The sorbent cartridge according to Clause 4, configured such that when in use, the dialysis liquid passes through the layer comprising zirconium phosphate before passing through the layer comprising hydrous zirconium oxide.

6. The sorbent cartridge according to any one of the preceding Clauses, wherein the difference in the moisture content of the first layer and the second layer is less than or equal to 5 wt. %.

7. The sorbent cartridge according to Clause 6, wherein the difference in the moisture content of the first layer and the second layer is less than or equal to 4 wt. %, optionally less than or equal to 3 wt. %.

8. The sorbent cartridge according to Clause 4 or 5, wherein the difference in the moisture content of the second layer and the third layer is less than or equal to 5 wt. %.

9. The sorbent cartridge according to Clause 8, wherein the difference in the moisture content of the second layer and the third layer is less than or equal to 4 wt. %, optionally less than or equal to 3 wt. %.

10. The sorbent cartridge according to any one of Clauses 4, 5, 8 and 9, wherein the difference between:
the moisture content of the first layer; and
the weighted average of the moisture contents of the second and third layers,
is less than or equal to 8 wt. %, optionally less than or equal to 5 wt. %, more optionally less than or equal to 4 wt. %, such as less than or equal to 3 wt. %,
wherein the weighted average of the moisture contents of the second and third layers is based on the mass of hydrous zirconium oxide and mass of zirconium phosphate.

11. The sorbent cartridge according to any one of the preceding Clauses, wherein the first layer has a moisture content of from 20-35 wt. %, optionally from 23-30 wt. % or from 25-35 wt. %.

12. The sorbent cartridge according to any one of the preceding Clauses, wherein the second layer has a moisture content of from 20-35 wt. %, optionally from 23-30 wt. % or from 25-35 wt.

13. The sorbent cartridge according to any one of Clauses 4, 5 and 8 to 12, wherein the third layer has a moisture content of from 20-35 wt. %, optionally from 23-30 wt. % or from 25-35 wt.

14. The sorbent cartridge according to Clause 4, or according to any one of Clauses 5 and 8-13 as dependent on Clause 4, wherein the layer comprising hydrous zirconium oxide has a moisture content of from 25-35 wt. %.

15. The sorbent cartridge according to Clause 3, or according to any one of Clauses 6, 7, 11, 12 or 13 as dependent on Clause 3, wherein the moisture content of the second layer comprising a homogenous mixture of hydrous zirconium oxide and zirconium phosphate is from 23-30 wt. %.

16. The sorbent cartridge according to any one of the preceding Clauses, further comprising a urease layer comprising immobilised urease.

17. The sorbent cartridge according to Clause 16, wherein the urease layer has a moisture content of from 17-27 wt. %, such as 19-25 wt. %, e.g. 20-24 wt. %.

18. The sorbent cartridge according to Clause 16 or 17, wherein the urease layer is in direct contact with at least one of the first layer, the second layer and, when present, the third layer, where the difference in the moisture content of the urease layer and the layers with which the urease layer is in direct contact is less than or equal to 8 wt. %, such as less than or equal to 5 wt. %, less than or equal to 4 wt. % or less than or equal to 3 wt. %, optionally wherein when the sorbent cartridge is configured such that when in use, the urease layer is located upstream from a layer comprising zirconium phosphate.

19. The sorbent cartridge according to any one of the preceding Clauses, wherein the moisture contents of the first, second, and when present, third and urease, layers are as determined by loss on drying.

20. The sorbent cartridge according to Clause 19, wherein the moisture contents of the first, second, and when present, third and urease, layers are as determined by loss on drying using a Radwag MA200.3Y moisture analyser at 180-220° C., optionally at about 210° C.

### BRIEF DESCRIPTION OF THE FIGURES

FIG. 1 shows an exemplary structure of a three-layered sorbent.

FIG. 2 shows an exemplary structure of a two layered sorbent.

FIG. 3 shows the results of the storage stability test of Comparative Example 1.

FIG. 4 shows the results of the storage stability test of Example 1.

FIG. 5 shows the results of the storage stability test of Comparative Example 2.

FIG. 6 shows the results of the storage stability test of Example 2.

### DETAILED DESCRIPTION OF THE INVENTION

As mentioned herein, the binding capacity of ion exchanging materials in sorbents (e.g. zirconium phosphate and hydrous zirconium oxide) is dependent on the moisture content of the ion exchanging material, as shown in Table 1.

TABLE 1
binding capacity of ion exchanging materials zirconium
phosphate and hydrous zirconium oxide

| ZP (Ammonia Binding) | | HZO (Phosphate Binding) | |
|---|---|---|---|
| Loss on drying | Binding capacity | Loss on drying | Binding capacity |
| 21.69% | 1.05 mmol/g | 37.26% | 0.63 mmol/g |
| 5.60% | 0.32 mmol/g | 9.30% | 0.35 mmol/g |

Activated carbon is a common sorbent component, and is commercially available with a moisture content of approximately 8 wt. %. This is drastically different to the preferred moisture contents for ZP and HZO when used in peritoneal dialysis. Without wishing to be bound by theory, it is believed by the current inventors that a sorbent formed from layers of commercially available AC, along with ZP and HZO of the desired moisture level is liable to crack or separate upon longer term storage (Comparative Examples 1 and 2). The terms “crack” and “separate” as used in this context are intended to refer to gaps or air pockets that form within a previously compact solid powder, whether vertically, horizontally, or any angle in between. These gaps or air pockets may form between different layers of the sorbent, or within a layer of the sorbent,

The invention solves the problems associated with the prior art. Thus, the invention provides a sorbent cartridge comprising:
a first layer comprising activated carbon; and
a second layer in direct contact with the first layer, the second layer comprising
one or both of hydrous zirconium oxide and zirconium phosphate,

wherein:
the first layer has a moisture content of X wt. %;
the second layer has a moisture content of Y wt. %; and
the difference in the moisture content of the first layer and the second layer is less than or equal to 8 wt. %.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

As used herein, a “sorbent cartridge” refers to a container that comprises materials that are able to remove toxins from a dialysate fluid, i.e. a kidney dialysis sorbent cartridge. More particularly, the kidney dialysis sorbent cartridge may be suitable for used in peritoneal dialysis.

The sorbent cartridge of the invention comprises at least two layers: a first layer comprising activated carbon; and a second layer comprising one or both of hydrous zirconium oxide and zirconium phosphate. The second layer is in direct contact with the first layer.

The first and second layers both have a moisture content, denoted herein as X wt. % and Y wt. %, respectively. The difference between the moisture content of the first and second layers is less than or equal to 8 wt. %. In other words, the difference between X and Y is less than or equal to 8.

As used herein, the “moisture content” of a layer refers to the amount of adsorbed or absorbed liquid present in the solid components of a layer. Typically, the moisture content will be predominantly composed of water, but other liquids may also form part of the overall moisture content. In some embodiments of the invention that may be mentioned herein, the moisture content of a layer may refer to the water content of said layer. In some embodiments of the invention that may be mentioned herein, the moisture content of a layer is the same as the loss on drying of that layer, for example the loss on drying at 210° C. of that layer (e.g. as measured by a Radwag MA200.3Y moisture analyser at 210° C.). For example, a solid sample (e.g. 1-2 g) may be placed within a Radwag MA200.3Y moisture analyser and a loss on drying experiment may be performed at 210° C. This experiment involves heating the sample at 210° C. until the instrument determines the loss on drying.

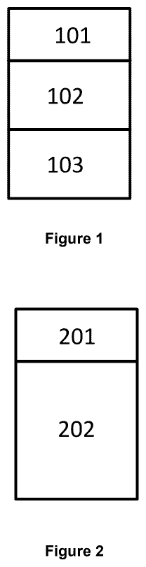

In some embodiments of the invention that may be mentioned herein, the second layer may comprise hydrous zirconium oxide. The sorbent cartridge may then comprise a third layer comprising zirconium phosphate. An example of one such embodiment is depicted graphically in FIG. 1, in which 101 represents activated carbon, 102 represents hydrous zirconium oxide and 103 represents zirconium phosphate.

In some embodiments of the invention that may be mentioned herein, the second layer may comprise zirconium phosphate. The sorbent cartridge may then comprise a third layer comprising hydrous zirconium oxide. These embodiments correspond to that shown in FIG. 1 in which layers 102 and 103 are reversed.

When present, the third layer has a moisture content of Z wt. %, and may be in direct contact with the second layer. In such embodiments of the invention, the difference in moisture content between the second layer and the third layer may be less than or equal to 8 wt. %. In other words, the difference between Y and Z may be less than or equal to 8.

In embodiments of the invention in which the sorbent cartridge comprises separate layers of zirconium phosphate and hydrous zirconium oxide, when in use, the dialysis liquid may pass through the layer comprising zirconium phosphate before passing through the layer comprising hydrous zirconium oxide. Thus, in the embodiment shown in FIG. 1, dialysis liquid may be passed through layer 103 first, before passing through layer 102 and finally layer 101. If layers 102 and 103 are reversed such that zirconium phosphate is present in the middle layer, then the dialysis liquid may first pass through the activated carbon layer, before passing through the zirconium phosphate middle layer and finally the hydrous zirconium oxide layer.

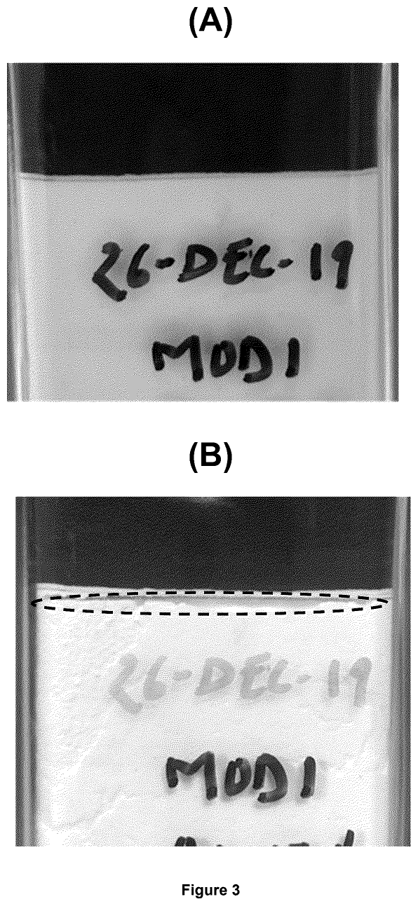

In some embodiments of the invention that may be mentioned herein, the second layer may comprise hydrous zirconium oxide and zirconium phosphate (e.g. a homogeneous mixture of hydrous zirconium oxide and zirconium phosphate). An example of one such embodiment is depicted graphically in FIG. 2, in which 201 represents activated carbon, and 202 represents a layer comprising both hydrous zirconium oxide and zirconium phosphate.

In embodiments of the invention that may be mentioned herein, the difference in moisture content between adjacent layers (e.g. the first and second layers, or the second and third layers) may be less than or equal to 8 wt. %. In further embodiments of the invention that may be mentioned herein, the difference in moisture content between adjacent layers may be less than or equal to 5 wt. %, 4 wt. % or 3 wt. %. As will be appreciated by a person skilled in the art, these values apply equally to the difference in moisture content between the first and second layers, and also between the second and third layers. All conceivable combinations of difference in moisture content across layers are explicitly contemplated herein.

In embodiments of the invention in which the second layer comprises one of zirconium phosphate and hydrous zirconium oxide, and the third layer comprises the other of zirconium phosphate and hydrous zirconium oxide, the difference between:
the moisture content of the first layer; and
the weighted average of the moisture contents of the second and third layers,
   may be less than or equal to 8 wt. % (for example less than or equal to 5 wt. %, e.g. less than or equal to 4 wt. %, such as less than or equal to 3 wt. %). In such embodiments, the weighted average of the moisture contents of the second and third layers is based on the mass of hydrous zirconium oxide and mass of zirconium phosphate in these layers.

In some embodiments of the invention that may be mentioned herein, the low difference in moisture content between the first and second layers may be obtained by using an activated carbon layer (first layer) having a moisture content that is higher than commercially available activated carbon. By increasing the moisture content of the activated carbon, a small difference in moisture content between layers may be achieved without significantly reducing the moisture content of hydrous zirconium oxide and zirconium phosphate. In this way, a small difference in moisture content across layer boundaries may be achieved without compromising the ability of the ion exchanging compounds (hydrous zirconium oxide and zirconium phosphate) to exchange ions. In contrast, if the moisture content of the hydrous zirconium oxide and zirconium phosphate is too low, then the ion exchanging ability may be compromised as shown in Table 1 above.

In some embodiments of the invention that may be mentioned herein, the first layer may have a moisture content of from 20-35 wt. %, e.g. from 23-30 wt. % or from 25-35 wt. %. As will be appreciated by a person skilled in the art, when the first layer has a moisture content according to this embodiment, the difference in the moisture content of the first layer and the second layer is less than or equal to 8 wt. % (e.g. the second layer may have a moisture content of from 12-43 wt. %). In some aspects of this embodiment of the invention, the second layer may also have moisture content of from 20-35 wt. %, e.g. from 23-30 wt. % or from 25-35 wt. %.

In some embodiments of the invention that may be mentioned herein, the second layer may have a moisture content of from 20-35 wt. %, e.g. from 23-30 wt. % or from 25-35 wt. %. As will be appreciated by a person skilled in the art, when the second layer has a moisture content according to this embodiment, the difference in the moisture content of the first layer and the second layer is less than or equal to 8 wt. % (e.g. the first layer may have a moisture content of from 12-43 wt. %). In some aspects of this embodiment of the invention, the first layer may also have moisture content of from 20-35 wt. %, e.g. from 23-30 wt. % or from 25-35 wt. %.

In some embodiments of the invention that may be mentioned herein, the third layer, when present, may have a moisture content of from 20-35 wt. %, optionally from 23-30 wt. % or from 25-35 wt. %. As will be appreciated by a person skilled in the art, when the third layer has a moisture content according to this embodiment, the difference in the moisture content of the second layer and the third layer is less than or equal to 8 wt. % (e.g. the second layer may have a moisture content of from 12-43 wt. %). In some aspects of this embodiment of the invention, the second layer may also have moisture content of from 20-35 wt. %, e.g. from 23-30 wt. % or from 25-35 wt. %.

In embodiments of the invention that comprise separate layers of zirconium phosphate and hydrous zirconium oxide that may be mentioned herein, the layer comprising hydrous zirconium oxide may have a moisture content of from 25-35 wt. %.

In embodiments of the invention where the second layer comprises both zirconium phosphate and hydrous zirconium oxide (e.g. a homogeneous mixture of zirconium phosphate and hydrous zirconium oxide) that may be mentioned herein, the moisture content of the second layer comprising a homogenous mixture of hydrous zirconium oxide and zirconium phosphate may be from 23-30 wt. %.

The wt. % values mentioned above are believed to be advantageous because they will avoid cracks or separation between layers whilst still providing sufficient moisture content for the zirconium phosphate and hydrous zirconium oxide to effectively exchange ions during the dialysis process.

As will be appreciated by a person skilled in the art, the sorbent cartridge of the invention may comprise additional layers. For example, in some embodiments of the invention that may be mentioned herein the sorbent cartridge may comprise a urease layer comprising immobilised urease. In some such embodiments, the urease layer may have a moisture content of from 17-27 wt. %, such as 19-25 wt. %, e.g. 20-24 wt. %.

In embodiments of the invention in which a urease layer is present, the urease layer may be in direct contact with at least one of the first layer, the second layer and, when present, the third layer. The difference in the moisture content of the urease layer and the layers with which the urease layer is in direct contact may be less than or equal to 8 wt. %, such as less than or equal to 5 wt. %, less than or equal to 4 wt. % or less than or equal to 3 wt. %.

In embodiments of the invention in which a urease layer is present, the urease layer is preferably located in an upstream layer as compared to a layer comprising zirconium phosphate. This is because the urease layer will generate ammonia, which should be removed from the fluid passing through the sorbent so that it is not passed to the patient. The ammonia may be removed by zirconium phosphate, and accordingly the urease layer is preferably located upstream from the zirconium phosphate layer.

The invention is illustrated by the below Examples, which are not to be construed as limitative.

### EXAMPLES

### Abbreviations

AC=activated carbon
ZP=zirconium phosphate
HZO=hydrous zirconium oxide
LOD=loss on drying

All materials were obtained from commercial suppliers and used without further purification.

### General Methods

In the below Examples and Comparative Examples, layered sorbents were prepared according to the following methodology.

Layer 1 (activated carbon) having a loss on drying (LOD) of 8-10% was obtained from a commercial supplier.

Layer 1 (activated carbon) having LOD of 20-25% was prepared by adjusting the moisture content of AC. De-ionized water was added to AC having 8% LOD, the mixture was stirred using an overhead stirrer at 25° C. for 5-10 minutes. LOD was then measured on a Radwag MA200.3Y moisture analyser at 210° C.

In order to obtain Activated carbon of LOD 25% from 100 g of Activated carbon of LOD 8%, below formula was used

$$\frac{( 8 + x )}{100 + x} = \frac{25}{100}$$

where, x=amount of water (g) required to be added to 100 g of AC of LOD 8% to make AC of LOD of 25%

Layers comprising zirconium phosphate and hydrous zirconium oxide were prepared by mixing ZP and HZO at specified quantities. ZP having a LOD 20-25% and HZO having a LOD 35-40% were obtained from a commercial supplier. HZO having a LOD of 25% was obtained by heating commercially supplied HZO of known LOD until the necessary loss had occurred.

Loss on drying was analysed as follows. A solid sample (e.g. 1-2 g) was be placed within a Radwag MA200.3Y moisture analyser and a loss on drying experiment was performed at 210° C. This experiment involved heating the sample at 210° C. until the instrument determines the loss on drying.

### Comparative Example 1

A two-layer Comparative Sorbent 1 was prepared with the layers in Table 2 below.

TABLE 2
Comparative Sorbent 1

| Layer | Moisture | Amount |
|---|---|---|
| AC (Layer 1) | 13% | 68 g |
| ZP + HZO (Layer 2) | ~25% | 325.2 g |
| | (20% ZP, | (256.8 g ZP, |
| | 37% HZO) | 68.4 g HZO) |

Comparative Sorbent 1 is a conventional sorbent in which the different layers have typical moisture levels known in the art. The difference in moisture content between layers 1 and 2 is approximately 12%.

Photographs of the results of a 9 week storage test at ambient conditions/room temperature are shown in FIG. 3, in which (A) shows Comparative Sorbent 1 at week 0, and (B) shows Comparative Sorbent 1 at week 9. The black portion of the Figure is the AC layer, while the white portion is the ZP/HZO layer.

After 9 weeks, visible cracks and non-uniformity had formed between the layers, as shown within the dashed lines. Comparative Sorbent 1 was therefore not storage stable for 9 weeks.

### Example 1

A two-layer Sorbent 1 was prepared with the layers in Table 3 below.

TABLE 3
Sorbent 1

| Layer | Moisture | Amount |
|---|---|---|
| AC (Layer 1) | 25% | 89 g |
| ZP + HZO (Layer 2) | 20-25% | 325.2 g |
| | (20% ZP, | (256.8 g ZP, |
| | 25% HZO) | 68.4 g HZO) |

Sorbent 1 is a sorbent according to the invention in which the difference in moisture levels between the two layers is minimised. The difference in moisture content between layers 1 and 2 is from 0-5%.

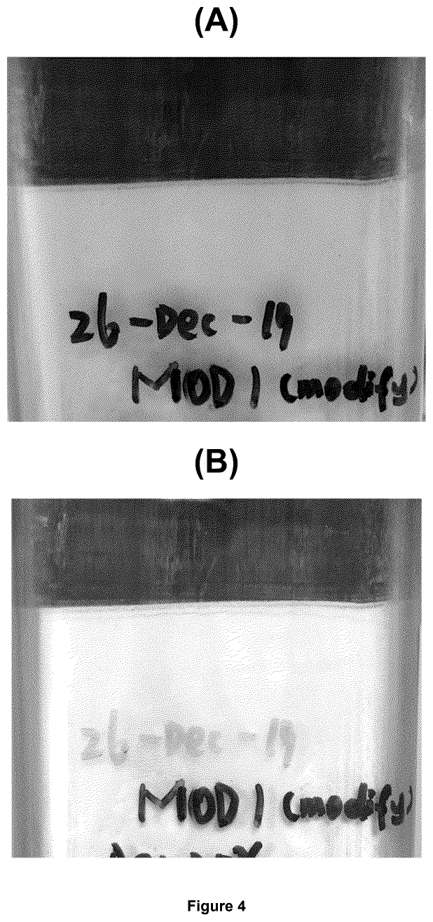

Photographs of the results of a 9 week storage test at ambient conditions/room temperature are shown in FIG. 4, in which (A) shows Sorbent 1 at week 0, and (B) shows Sorbent 1 at week 9. The black portion of the Figure is the AC layer, while the white portion is the ZP/HZO layer.

There were no visible cracks or other non-uniformity between the layers after 9 weeks.

Sorbent 1 was therefore shown to be storage stable under ambient conditions for at least 9 weeks.

### Comparative Example 2

Comparative Sorbent 2 was prepared according to Table 4 below.

TABLE 4
Comparative Sorbent 2

| Layer | Moisture | Amount |
|---|---|---|
| AC (Layer 1) | 8% | 81 g |
| ZP + HZO (Layer 2) | 25-28% | 340 g |
| | (20-23% ZP, | (187 g ZP, |
| | 37-40% HZO) | 153 g HZO) |

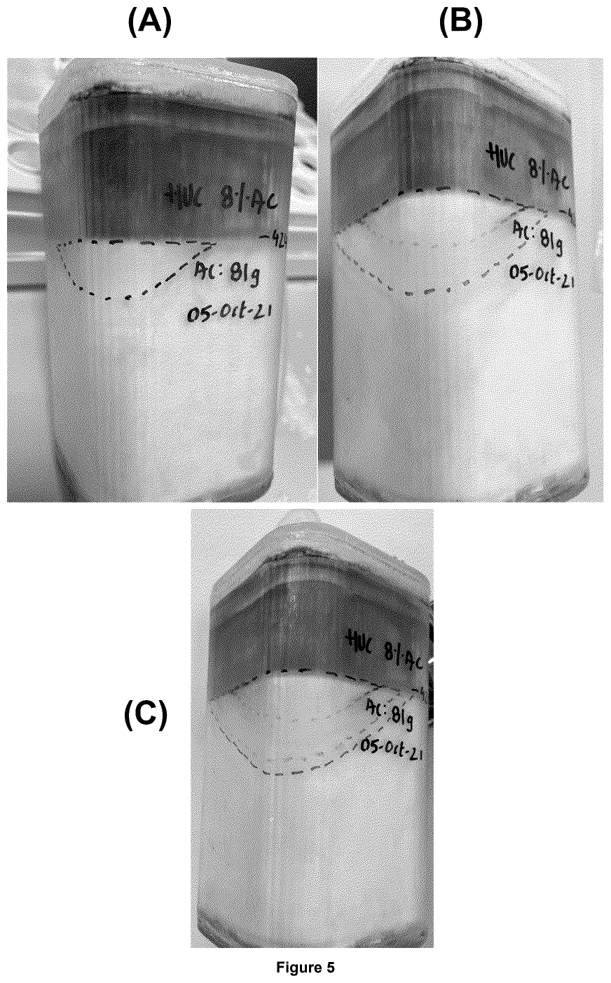

The difference in moisture content between layers 1 and 2 is approximately 17-20% Photographs of the results of an 8 week storage test at ambient conditions/room temperature are shown in FIG. 5. (A) shows the sorbent cartridge at 2 weeks, (B) shows the sorbent cartridge at 4 weeks and (C) shows the sorbent cartridge at 8 weeks. The black portion of the Figure is the AC layer, while the white portion is the ZP/HZO layer.

After 2 weeks and thereafter, imperfections having different moisture levels and appearance were visible extending from the boundary between Layers 1 and 2 into Layer 2 (ZP+HZO). The imperfections grew in size over the duration of the 8 week test. The boundary of the imperfections at each time period is shown by dashed lines in FIG. 4.

Comparative Sorbent 2 was therefore not storage stable for 2 weeks, let alone 8 weeks.

### Example 2

Sorbent 2 was prepared according to Table 5 below.

TABLE 5
Sorbent 2

| Layer | Moisture | Amount |
|---|---|---|
| AC (Layer 1) | 24-29% | 98 g |
| ZP + HZO (Layer 2) | 25-28% | 340 g |
| | (20-23% ZP, | (187 g ZP, |
| | 37-40% HZO) | 153 g HZO) |

The difference in moisture content between layers 1 and 2 is approximately 1-4%.

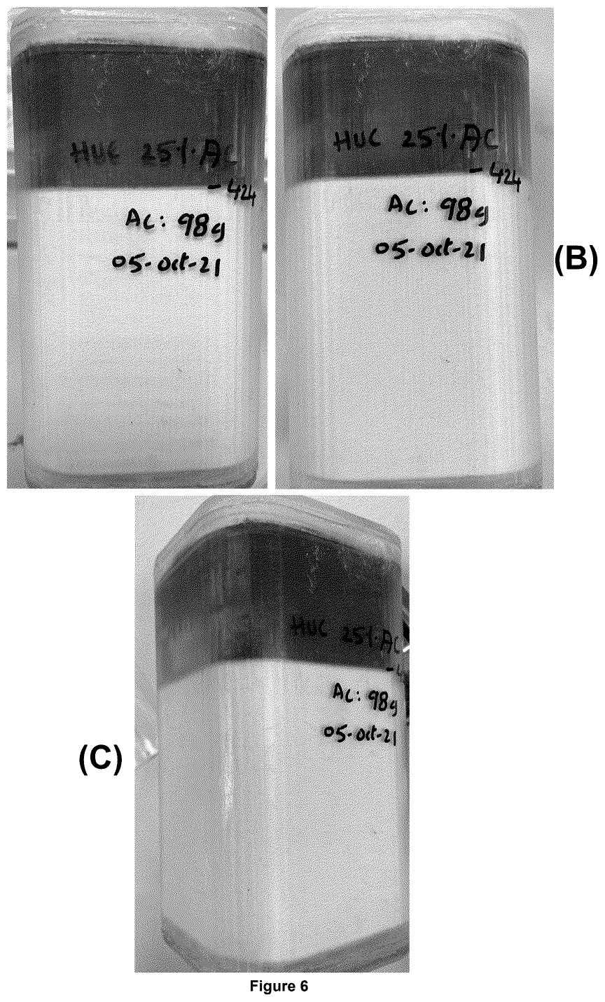

Photographs of the results of an 8 week storage test at ambient conditions/room temperature are shown in FIG. 6. (A) shows the sorbent cartridge at 2 weeks, (B) shows the sorbent cartridge at 4 weeks and (C) shows the sorbent cartridge at 8 weeks. The black portion of the Figure is the AC layer, while the white portion is the ZP/HZO layer.

No imperfections, separation or cracks between layers were observed over the duration of the 8 week test.

Sorbent 2 was therefore shown to be storage stable under ambient conditions for at least 8 weeks.

The above Examples and Comparative Examples demonstrate that the sorbent cartridge of the invention has excellent long term storage stability, and has improved stability as compared to conventional dialysis sorbent cartridges in which the difference in moisture content across a layer boundary is not controlled.

What is claimed:

1. A sorbent cartridge comprising:
a first layer comprising activated carbon; and
a second layer in direct contact with the first layer, the second layer comprising one or both of hydrous zirconium oxide and zirconium phosphate,
wherein:
the first layer has a moisture content of X wt. %;
the second layer has a moisture content of Y wt. %; and the difference in the moisture content of the first layer and the second layer is less than or equal to 8 wt. %.

2. The sorbent cartridge according to claim 1, wherein the second layer comprises both hydrous zirconium oxide and zirconium phosphate.

3. The sorbent cartridge according to claim 2, wherein the second layer comprises a homogenous mixture of hydrous zirconium oxide and zirconium phosphate.

4. The sorbent cartridge according to claim 1, wherein:
the second layer comprises one of zirconium phosphate and hydrous zirconium oxide;
the sorbent cartridge further comprises a third layer comprising the other of zirconium phosphate and hydrous zirconium that is not present in the second layer;
the third layer has a moisture content of Z wt. %; and
the difference in the moisture content of the second layer and the third layer is less than or equal to 8 wt. %.

5. The sorbent cartridge according to claim 4, configured such that when in use, the dialysis liquid passes through the layer comprising zirconium phosphate before passing through the layer comprising hydrous zirconium oxide.

6. The sorbent cartridge according to claim 1, wherein the difference in the moisture content of the first layer and the second layer is less than or equal to 5 wt. %.

7. The sorbent cartridge according to claim 6, wherein the difference in the moisture content of the first layer and the second layer is less than or equal to 4 wt. %.

8. The sorbent cartridge according to claim 4, wherein the difference in the moisture content of the second layer and the third layer is less than or equal to 5 wt. %.

9. The sorbent cartridge according to claim 8, wherein the difference in the moisture content of the second layer and the third layer is less than or equal to 4 wt. %.

10. The sorbent cartridge according to claim 4, wherein the difference between:
the moisture content of the first layer; and
the weighted average of the moisture contents of the second and third layers,
is less than or equal to 8 wt. %,
wherein the weighted average of the moisture contents of the second and third layers is based on the mass of hydrous zirconium oxide and mass of zirconium phosphate.

11. The sorbent cartridge according to claim 1 wherein the first layer has a moisture content of from 20-35 wt. %.

12. The sorbent cartridge according to claim 1, wherein the second layer has a moisture content of from 20-35 wt. %.

13. The sorbent cartridge according to claim 4, wherein the third layer has a moisture content of from 20-35 wt. %.

14. The sorbent cartridge according to claim 4, wherein the layer comprising hydrous zirconium oxide has a moisture content of from 25-35 wt. %.

15. The sorbent cartridge according to claim 3, wherein the moisture content of the second layer comprising a homogenous mixture of hydrous zirconium oxide and zirconium phosphate is from 23-30 wt. %.

16. The sorbent cartridge according to claim 1, further comprising a urease layer comprising immobilised urease.

17. The sorbent cartridge according to claim 16, wherein the urease layer has a moisture content of from 17-27 wt. %.

18. The sorbent cartridge according to claim 16, wherein the urease layer is in direct contact with at least one of the first layer, the second layer and, when present, the third layer, where the difference in the moisture content of the urease layer and the layers with which the urease layer is in direct contact is less than or equal to 8 wt. %.

19. The sorbent cartridge according to claim 1, wherein the moisture contents of the first and second layers are as determined by loss on drying.

20. The sorbent cartridge according to claim 1, wherein the moisture contents of the first and second are as determined by loss on drying using a Radwag MA200.3Y moisture analyser at 180-220° C.

21. The sorbent cartridge according to claim 18, wherein the sorbent cartridge is configured such that when in use, the urease layer is located upstream from a layer comprising zirconium phosphate.
